(19) United States
(12) Patent Application Publication
(10) Pub. No.: US 2022/0371813 A1
(43) Pub. Date: Nov. 24, 2022

(54) ONE-DIRECTION GLOVE DISPENSER

(71) Applicant: CRANBERRY INTERNATIONAL SDN BHD, Shah Alam, Selangor (MY)

(72) Inventors: Devisri Shobana M K MATHAVAN, Shah Alam, Selangor (MY); Shu Theng LIM, Shah Alam, Selangor (MY)

(21) Appl. No.: 17/624,211

(22) PCT Filed: Oct. 14, 2019
(86) PCT No.: PCT/MY2019/050072

§ 371 Date: Dec. 30, 2021

(51) Int. Cl. B65D 83/08; B65D 85/18; B65B 35/50; B65B 5/06; A61B 42/40

(52) U.S. Cl. CPC B65D 83/0823; B65D 83/0894; B65D 85/18; B65B 35/50; B65B 5/06; A61B 42/40; A41D 19/0055

(57) ABSTRACT

The present invention provides a gloves dispensing device (105) comprising: a plurality of gloves (101); a container for accommodating said gloves (101); characterised in that the container having an aperture (107) for dispensing said gloves by a user, wherein the gloves (101) are stacked unidirectional in which at least one area of cuffs (104) of the gloves are exposed within the aperture (107) to reduce exposure of the external surfaces of finger (102) portions of said gloves (101).

### TECHNICAL FIELD

The present invention generally relates to a dispensing device for latex or synthetic gloves employed for common procedures performed in medical and non-medical environment, as well as in household applications.

### BACKGROUND OF THE INVENTION

A dispensing device typically refers to a container which is designed to dispense a specific product as needed by a user, by opening a cover of an aperture. A tissue dispensing device is generally a rectangular enclosure with a top opening, and often made of durable cardboard or plastic to retain its shape while protecting the tissue housed within. The covering of the aperture of said dispensing device may be a part that is not separate from the construction of said device (therefore of a similar material), a peelable paper or aluminium adhesive, and a hinged plastic covering or lid. The aperture itself is usually covered with a thin shield of plastic film with a cut in the middle of the aperture to retain the contents within the box without clumping at said aperture.

The use of latex or synthetic gloves is one of the common primary preventive measures that are taken by both medical and non-medical specialists and scientists to reduce contamination in sensitive environment. Cross-contamination occurs due to improper glove dispensing mechanism, in which conventional glove dispensing containers has an aperture that immediately leaves the gloves exposed to the air particle once the opening is uncovered.

When worn, latex or synthetic gloves provide a barrier between the hands of a user and the object of operation, thus the hands of said user and object are not in direct contact with each other. This minimises the transfer of contaminants between the two provided that the latter are used in conjunction with meticulous cleaning techniques, and are not perforated during handling.

The user usually pulls a glove, or more if the gloves are clumped, from a glove dispensing box using uncovered hands. The gloves are now no longer sanitary since human hands and the surrounding air are prolific agents of bioburden, typically comprising of microflora such as bacteria and fungi. If the gloves are pulled in clumps, more gloves are soiled with contaminants. This leads to unnecessary wastage, leading to less uncontaminated gloves available for use at a given time. Therefore, an alternative invention to overcome this problem is required.

### SUMMARY OF THE INVENTION

One embodiment of the present invention relates to a gloves dispensing device comprising: a plurality of gloves; a container for accommodating said gloves; characterised in that the container having an aperture for dispensing said gloves by a user, wherein the gloves are stacked unidirectional in which at least one area of cuffs of the gloves are exposed within the aperture to reduce exposure of the external surfaces of finger portions of said gloves.

Further, the gloves dispensing device reduced exposure of the external surfaces of finger portions reduces cross contamination of the finger portions.

Preferably, the gloves stacked unidirectional reduces clumping said gloves.

Preferably, the gloves are stacked unidirectional, eases the dispensing of the gloves.

Preferably, the aperture for dispensing said gloves is covered by a tearing-off flap or a flap with cut-out or perforated portion.

Preferably, the aperture is positioned one end of the dispensing device surface area, wherein the dispensing device is rectangular or box shape.

Preferably, the aperture is in teardrop-shape, triangle, trapezium, rectangular, oval, or semicircle like shape.

Preferably, the aperture positioned on one end surface of the gloves dispensing device to accommodate proper branding or providing clear indication of product details and specification on the other end of the surface of the gloves dispensing device.

Preferably, the container or dispensing device having a lifting mechanism formed at the bottom of the container, for pushing up gloves stacked within the container.

Preferably, the lifting mechanism include having at least one pair of flaps formed at base or bottom of the container that able to be pushed up to lift up the gloves resting on the bottom of the container, to ease the access of dispensing the gloves.

Preferably, the lifting mechanism is perforated or has a cut-out portion, which is pushed up by at least one user to position the gloves in the container at desirable or closer to the aperture perforation.

Alternative embodiment of the present invention relates to a gloves dispensing device comprising: a plurality of gloves; a container for accommodating said gloves; characterised in that the container having the gloves are stacked unidirectional in which at least one area of cuffs portion of the gloves are positioned below at least one aperture for dispensing, such that finger portions of said gloves is covered within the container when a user is dispensing the gloves.

Further, the plurality of gloves are assembled in such a way that only the external cuffs of the gloves are exposed to the aperture to reduce cross-contamination between the external surfaces of finger portions of said gloves and a user.

Preferably, the gloves are stacked unidirectionally such that when arranged into a carton, the aperture of the dispensing device is positioned upwards in line with cuffs of the gloves, while finger portions of said gloves is positioned downwards to reduce clumping of the finger.

Preferably, the dispensing device positioned in upright position to align the gloves in position within the dispensing device for space saving arrangement.

Another embodiment of the present invention relates to a method of packing a gloves dispensing device, comprising steps of: forming an aperture for dispensing said gloves by a user on one end surface of the gloves dispensing device; stacking a plurality of gloves in unidirectional in which cuffs, finger and palm portions of the glove are in line; positioning the cuffs portions below the aperture within the gloves dispensing device; placing the gloves dispensing device into a carton, wherein the aperture of the dispensing device is positioned upwards, such that finger portions of said gloves is positioned downwards.

Further, the gloves are stacked unidirectional or positioned upwards to reduce clumping of the finger portions.

Preferably, the positioning the cuffs portions below the aperture reduces cross contamination of the external surfaces of finger portions.

Preferably, the aperture positioned on one end surface of the gloves dispensing device to accommodate proper branding or providing clear indication of product details and specification on the other end of the surface of the gloves dispensing device.

Preferably, the container or dispensing device has a lifting mechanism formed at the bottom of the container, for pushing up gloves stacked within the container.

Preferably, the lifting mechanism include having at least one pair of flaps formed at base or bottom of the container that able to be pushed up to lift up the gloves resting on the bottom of the container, to ease the access of dispensing the gloves.

Preferably, the lifting mechanism is perforated or has a cut-out portion, which is pushed up by at least one user to position the gloves in the container at desirable or closer to the aperture perforation.

The present invention consists of features and a combination of parts hereinafter fully described and illustrated in the accompanying drawings, it being understood that various changes in the details may be made without departing from the scope of the invention or sacrificing any of the advantages of the present invention.

### BRIEF DESCRIPTION OF THE DRAWINGS

To further clarify various aspects of some embodiments of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the accompanying drawings in which:

FIG. 1 is a top planar view of an embodiment of a plurality of latex or synthetic gloves contained within a dispensing device;

FIG. 2 is a cross-sectional view of an embodiment of a dispensing device containing a plurality of cuffs exposed to an uncovered aperture and;

FIG. 3 illustrates the assembly of a dispensing device into a carton.

### DETAILED DESCRIPTION OF THE PREFERRED EMBODIMENTS

In light of the aforementioned problem associated with prior glove dispensing devices, the present invention is to provide a dispensing device for latex or synthetic gloves, wherein the construct of the invention is in such a way that reduces clumping of said gloves upon dispensing, and significantly reduces cross-contamination between a user and said gloves. The aperture covering in which the gloves are dispensed from are perforated. The gloves are assembled within the dispenser in such a way that only the external cuffs of the glove bodies face the aperture covering. This ensures that the external surfaces of the finger portions of a glove body do not exposed to contamination during the dispensing stage. This invention reduces the frequency of fingers or gloves clumping upon dispensing from said dispensing device, and exposure of the fingers to reduce cross-contamination.

Further, the aperture perforation and aperture covering are shaped in such a way that they have one sharp corner for a user to initiate the peeling off of said covering. The shape of the aperture is preferably having a wider opening on the cuff area than the palm or finger area of the glove placed in the container. The opening on the container has sufficient width for at least one user to access the glove within the container using at least two finger, wherein the fingers could be inserted or have access into the glove-containing space, further said glove dispensing aperture or opening also preferably adequately small in size to allow the user to reach his or her fingers there through to grasp the required glove. The shape of the opening could be in triangle, trapezium, rectangular, oval, or semicircle like shape, in which the cuff area has a wider opening than the palm area of the gloves stacked in the container.

Preferably, the aperture is positioned one end of the dispensing device surface area, wherein the dispensing device is rectangular or box shape. Further, the aperture positioned on one end surface of the gloves dispensing device also able to accommodate proper branding and providing clear indication of product details and specification on the other end of the surface of the gloves dispensing device. Further it also the positioning of product details on the dispensing device meets and comply with the local Food and Drug Administration regulations or standards.

Preferably, the container or dispensing device has a lifting mechanism formed at the bottom of the container, for pushing up gloves stacked within the container. The lifting mechanism is disposed under the gloves in the container for pushing up gloves.

The lifting mechanism include having at least one pair of flaps formed at base or bottom of the container that able to be pushed up to lift up the gloves resting on the bottom of the container, to ease the access of dispensing the gloves. The lifting mechanism is perforated or has a cut-out portion, which is pushed up by at least one user to position the gloves in the container at desirable or closer to the aperture perforation.

Preferably, the dispensing device is derived from either cellulose, plastic material, paper, foil, PET (polyester)/PE (Poly Ethylene) film laminated, or material used for packaging machines in order to pack wet wipes, swaps, lens cleaner.

Preferably, the dispensing device materials made of paper-processed materials are mainly made of pulps, such as virgin pulp and recycled pulp used.

The present invention discloses an embodiment of a glove dispensing box for use in medical and non-medical, auto industry, cleanroom, research, manufacturing, and Food and Beverages (F&B) industries, comprising: a plurality of latex or synthetic gloves which are stacked unidirectionally in an open planar position within a rectangular dispensing device, in which only the external cuffs of said gloves are exposed to the aperture covering releasing the gloves to the user.

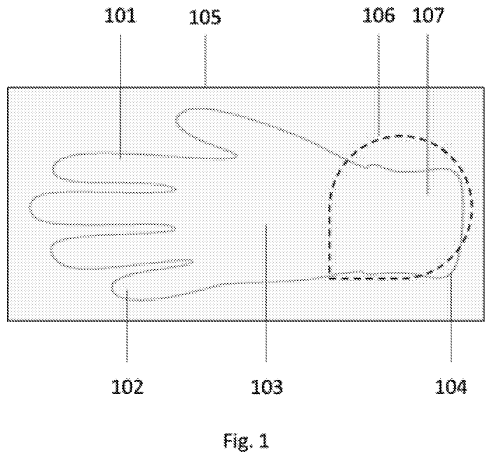

FIG. 1. shows an embodiment of a glove dispensing device (105) containing a plurality of latex or synthetic gloves (101) that are stacked on top of each other unidirectionally in an open planar position within an air-tight dispensing device (105) that is made of, but not limited to, either card or plastic material, whereby each glove is pulled out by a user through an opened aperture covering (107) that exposes only the external cuff (104) of said glove. The aperture covering (107) is uncovered by the tearing-off of an aperture perforation (106) that is teardrop-shaped or any other suitable shape. Said aperture covering (107) is also protected from contaminants between uses by a shield of thin plastic film with a middle incision. The aperture covering (107) is either placed on the right or left side of said dispensing device (105). The plurality of gloves (101) are assembled in such a way to reduce clumping when the user pulls out a glove as needed, and protect critical portions, that are the finger portions (102) of the latex or synthetic gloves (101) from contaminants during the dispensing process.

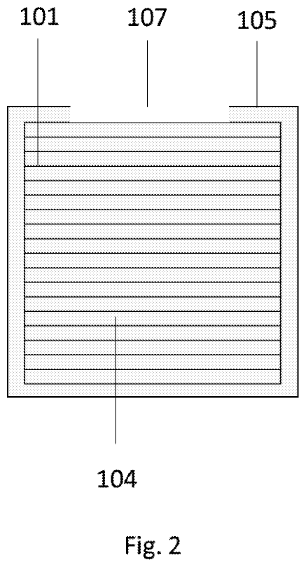

FIG. 2 shows a side cross-sectional view of an embodiment of a glove dispensing device (105) containing a plurality of latex or synthetic gloves (101), wherein the cuffs (104) are exposed to an opened to an aperture covering (107).

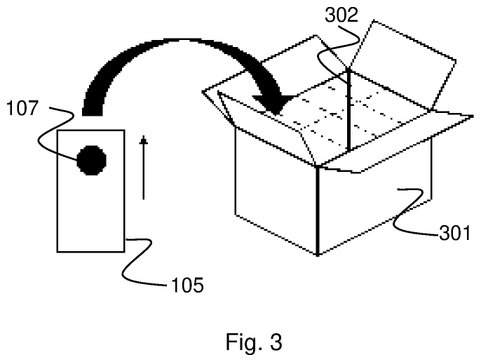

FIG. 3 shows a dispensing device (105) containing a plurality of latex or synthetic gloves is shown to be arranged into a carton (301) through an opening (302) of said carton, whereby the dispensing device (105) is arranged upwards with the aperture covering (107) facing the user at first contact. This ensures that the cuff portion of the plurality of gloves always faces the user at first contact. The finger portion of said gloves faces down in the dispensing container (105) in the above-mentioned arrangement, eliminating a problem of said finger portion rolling back towards the palm and cuff portion of the latex or synthetic gloves caused by gravity. An embodiment of said dispensing device (105) and gloves can be mounted either horizontally on flat surfaces, or vertically on wall frames that are designed to hold glove-dispensing devices. The shape of the dispensing device allows efficient utilisation of space whenever limited.

The present invention may be embodied in other specific forms without departing from its essential characteristics. The described embodiments are to be considered in all respects only as illustrative and not restrictive. The scope of the invention is, therefore indicated by the appended claims rather than by the foregoing description. All changes, which come within the meaning and range of equivalency of the claims, are to be embraced within their scope.

Throughout this specification, unless the context requires otherwise, the word “comprise”, or variations such as “comprises” or “comprising”, will be understood to imply the inclusion of a stated step or element or integer or group of steps or elements or elements or integers, but not the exclusion of any other step or element or integer or group of steps, elements or integers. Thus, in the context of this specification, the term “comprising” is used in an inclusive sense and thus should be understood as meaning “including principally, but not necessarily solely”.

1. A gloves dispensing device (105) comprising:
a plurality of gloves (101);
a container for accommodating said gloves (101);
characterised in that the container having an aperture (107) for dispensing said gloves by a user, wherein the gloves (101) are stacked unidirectional in which at least one area of cuffs (104) of the gloves are exposed within the aperture (107) to reduce exposure of the external surfaces of finger (102) portions of said gloves (101).

2. The gloves dispensing device (105) according to claim 1, wherein the reduced exposure of the external surfaces of finger (102) portions reduces cross contamination of the finger (102) portions.

3. The gloves dispensing device (105) according to claim 1, wherein the gloves (101) stacked unidirectional reduces clumping said gloves (101).

4. The gloves dispensing device (105) according to claim 1, wherein the gloves (101) are stacked unidirectional, eases the dispensing of the gloves (101).

5. The gloves dispensing device (105) according to claim 1, wherein the aperture (107) for dispensing said gloves is covered by a tearing-off flap or a flap with cut-out or perforated portion.

6. The gloves dispensing device (105) according to claim 1, wherein the aperture (107) is positioned one end of the dispensing device (105) surface area, wherein the dispensing device (105) is rectangular or box shape.

7. The gloves dispensing device (105) according to claim 1, wherein the aperture (107) is in teardrop-shape, triangle, trapezium, rectangular, oval, or semicircle like shape.

8. The gloves dispensing device (105) according to claim 1, wherein the aperture positioned on one end surface of the gloves dispensing device to accommodate proper branding or providing clear indication of product details and specification on the other end of the surface of the gloves dispensing device.

9. The gloves dispensing device (105) according to claim 1, wherein the container or dispensing device having a lifting mechanism formed at the bottom of the container, for pushing up gloves stacked within the container.

10. The gloves dispensing device (105) according to claim 9, wherein the lifting mechanism include having at least one pair of flaps formed at base or bottom of the container that able to be pushed up to lift up the gloves resting on the bottom of the container, to ease the access of dispensing the gloves.

11. The gloves dispensing device (105) according to claim 9, wherein the lifting mechanism is perforated or has a cut-out portion, which is pushed up by at least one user to position the gloves in the container at desirable or closer to the aperture perforation.

12. A gloves dispensing device (105) comprising:
a plurality of gloves (101);
a container for accommodating said gloves (101);
characterised in that the container having the gloves (101) are stacked unidirectional in which at least one area of cuffs (104) portion of the gloves are positioned below at least one aperture (107) for dispensing, such that finger (102) portions of said gloves (101) is covered within the container when a user is dispensing the gloves (101).

13. The gloves dispensing device (105) according to claim 8, wherein plurality of gloves (101) are assembled in such a way that only the external cuffs (104) of the gloves are exposed to the aperture (107) to reduce cross-contamination between the external surfaces of finger (102) portions of said gloves (101) and a user.

14. The gloves dispensing device (105) according to claim 8, wherein the gloves (101) are stacked unidirectional such that when arranged into a carton (301), the aperture (107) of the dispensing device (105) is positioned upwards in line with cuffs (104) of the gloves, while finger (102) portions of said gloves (101) is positioned downwards to reduce clumping of the finger (102) portions.

15. The gloves dispensing device (105) according to claim 10, wherein the dispensing device (105) positioned in upright position to align the gloves (101) in position within the dispensing device (105) for space saving arrangement.

16. A method of packing a gloves dispensing device (105), comprising steps of:
forming an aperture (107) for dispensing said gloves by a user on one end surface of the gloves dispensing device (105);
stacking a plurality of gloves (101) in unidirectional in which cuffs (104), finger (102) and palm (103) portions of the glove are in line;
positioning the cuffs (104) portions below the aperture (107) within the gloves dispensing device (105);
placing the gloves dispensing device (105) into a carton (301), wherein the aperture (107) of the dispensing device (105) is positioned upwards, such that finger (102) portions of said gloves (101) is positioned downwards.

17. The method of packing a gloves dispensing device (105) according to claim 16, wherein the gloves (101) are stacked unidirectional or positioned upwards to reduce clumping of the finger (102) portions.

18. The method of packing a gloves dispensing device (105) according to claim 16, wherein the positioning the cuffs (104) portions below the aperture (107) reduces cross contamination of the external surfaces of finger (102) portions.

19. The method of packing a gloves dispensing device (105) according to claim 16, wherein the aperture positioned on one end surface of the gloves dispensing device to accommodate proper branding or providing clear indication of product details and specification on the other end of the surface of the gloves dispensing device.

20. The method of packing a gloves dispensing device (105) according to claim 16, wherein the container or dispensing device having a lifting mechanism formed at the bottom of the container, for pushing up gloves stacked within the container.

21. The method of packing a gloves dispensing device (105) according to claim 20, wherein the lifting mechanism include having at least one pair of flaps formed at base or bottom of the container that able to be pushed up to lift up the gloves resting on the bottom of the container, to ease the access of dispensing the gloves.

22. The method of packing a gloves dispensing device (105) according to claim 20, wherein the lifting mechanism is perforated or has a cut-out portion, which is pushed up by at least one user to position the gloves in the container at desirable or closer to the aperture perforation.
